(19) United States
(12) Patent Application Publication
(10) Pub. No.: US 2009/0007912 A1
(43) Pub. Date: Jan. 8, 2009

(54) Ventilator for Supplying Breathable Gas to a Patient, and a Noise Reduction Method for Said Ventilator

(75) Inventors: Hans Lindell, Save (SE); Lars Ljungberg, Floda (SE); Staffan Bengtsson, Goteborg (SE); Johan Elgedin, Vrigstad (SE)

Correspondence Address: RAYMOND R. MOSER JR., ESQ.;MOSER IP LAW GROUP, 1030 BROAD STREET, 2ND FLOOR, SHREWSBURY, NJ, 07702, US

(73) Assignee: Breas Medical AB, Molnlycke (SE)

(21) Appl. No.: 10/599,675

(22) PCT Filed: Apr. 5, 2005
(86) PCT No.: PCT/SE2005/000494

§ 371 Date: Jul. 6, 2007

Related U.S. Application Data
(60) Provisional application No. 60/573,231, filed on May 21, 2004.

(30) Foreign Application Priority Data
Apr. 5, 2004 (SE) 0400892-6

(51) Int. Cl. A61M 16/00

(52) U.S. Cl. 128/204.18

(57) ABSTRACT

A ventilator for supplying breathable gas to a patient includes an external housing; an internal housing suspended within; a gas flow generator within said internal housing; a gas inlet conduit extending between a first gas inlet opening in said external housing and a second gas inlet opening in said internal housing, and a gas outlet conduit extending from a first gas outlet opening in the internal housing via a second gas outlet opening in the external housing and to a patient interface means adapted for introducing the breathable gas into the airway of said patient. One or both of the gas inlet and outlet conduits exhibit a first substantially rigid conduit section and a second membrane conduit section with a membrane wall portion separating a volume of breathable gas within the gas inlet conduit and/or gas outlet conduit from a volume of ambient air within the external housing.

### FIELD OF THE INVENTION

The present invention relates to a ventilator for supplying breathable gas, normally air, at elevated pressure to a patient for treating breathing disorders such as for example Obstructive Sleep Apnea (OSA), Cheyne-Stokes respiration or emphysema. The ventilator may also be used in the treatment of cardiac disorders, such as Congestive Heart Failure (CHF). The invention is applicable to advanced intensive care ventilators for assisted ventilation or to various types of Continuous Positive Airway Pressure ventilators (CPAP). More particularly, the ventilator comprises novel noise reduction features, that substantially reduce the operating noise level of the ventilator and allows a more compact design, thus improving user comfort for the patients. The invention also relates to a noise reduction method for said ventilator.

### BACKGROUND OF THE INVENTION

Ventilators for supplying breathable gas to the airway of a patient, are well known in the art per se. In simplest form of CPAP therapy, air of a constant positive pressure is supplied to the airway of a patient, in order to treat Obstructive Sleep Apnea (OSA). The required pressure level varies for individual patients and their respective breathing disorders. CPAP therapy may be applied not only to the treatment of breathing disorders, but also to the treatment of Congestive Heart Failure (CHF).

A more advanced form of CPAP therapy is commonly referred to as Bi-Level CPAP, wherein air is applied to the airway of a patient alternatively at a higher pressure level during inspiration and a lower pressure level during expiration. The higher pressure level is referred to as IPAP (Inspiratory Positive Airway Pressure), whilst the lower pressure level is referred to as EPAP (Expiratory Positive Airway Pressure). In a Bi-level CPAP ventilator, EPAP and IPAP are thus synchronized with the patient's inspiratory cycle and expiratory cycle so that the patient will not be forced to overcome a high pressure from the ventilator during the expiration phase of his or her breathing. Consequently, Bi-Level CPAP ventilators generally provides improved breathing comfort for the patient compared to the simpler “single level” CPAP ventilator described initially. In order to detect the patients transition from the inspiratory breathing phase to the expiratory breathing phase, a Bi-Level CPAP ventilator is provided with one or more sensors. Normally, a flow sensor is located somewhere along the air supply conduit to the patient. Additionally, a pressure sensor may for example be located in a patient interface means, such as a facial mask, or along the air supply conduit. The different pressure levels and/or flow levels are normally controlled by means of a control valve, which restricts and directs the airflow in various ways. As will be described in more detail below, modern ventilators often use a gas flow generator in the form of an electric fan unit, and the pressure and flow may thus be additionally or exclusively controlled by varying the rotary speed of the fan.

Another, yet more advanced type of CPAP ventilator is generally referred to as an AutoCPAP ventilator. Other terms for this type of ventilator include: Auto Adaptive CPAP (AACPAP), Auto Titration CPAP or Self-titrating individual AutoCPAP. In this description, these terms will commonly be referred to as an AutoCPAP ventilator for the sake of clarity. Here, IPAP and EPAP as well as other relevant parameters are automatically changed with respect to specific detected breathing patterns significative of different breathing disorders or phases thereof. This is an “intelligent” form of CPAP treatment, in which a certain condition may even be foreseen by the ventilator before the condition is felt by the patient, and wherein a suitable combination of IPAP and EPAP as well as other relevant parameters are applied in order to treat or alleviate the symptoms of the patient. For this purpose, it is known to provide a ventilator with an integral learning artificial neural network (ANN) to gather large amounts of relevant breathing data from a vast population of patients with breathing disorders worldwide. The ANN is able to detect and identify breathing patterns that are symptomatic of a certain condition or disorder and to then automatically adapt the ventilator parameter settings for effecting a relevant treatment pattern at an early stage. Apart from added control hardware, software and more sensors, the basic hardware design of an AutoCPAP ventilator may be substantially identical a Bi-Level CPAP ventilator.

A trend in modern ventilator technology is directed toward ever more compact and lightweight CPAP ventilators, that are unobtrusive at the bedside, offer increased mobility for patients and generally have a less “hospital-like” design, in order to improve user comfort.

All ventilator types described above each include a gas flow generator for creating a gas flow to the patient. A patient interface means, in the form of a facial mask or a tracheal tube is provided for introducing the breathable gas into the airway of the patient.

In older ventilators, the gas flow generator often consisted of an air bellows unit, which was normally comfortably quiet, but had to be rather large in order to effectively produce the required airflow. Thus, in modern, more compact ventilators, a compact but effective electrical fan unit has replaced the air bellows often found in older systems.

A problem with introducing electric fan units instead of air bellows is that they have to run at relatively high rotational speeds in order to produce the required airflow and still meet the modern requirements for a more compact overall design. This makes them inherently noisy, due to high frequency aero-dynamic noise generated by the fan rotor wings and the fan housing, and structurally borne noise due to rotor unbalance, electro-dynamic forces etc. Thus, although the electric fan unit enables a more compact design in itself, it also requires more noise reduction efforts in order to meet the requirements for a comfortable operational sound level. Conventionally, ventilators are muffled or noise reduced by lining the ventilator housing with sound absorbing padding, such as plastic foam. This practise effectively sets a lower physical limit to the overall size of the ventilator, since the thickness of the sound absorbing padding required to muffle a fan unit of this type will be a deciding factor.

### OBJECT OF THE INVENTION

It is the object of the present invention to enable an improved ventilator design that is quieter and yet even more compact than current ventilators, thus offering a highly improved user comfort for patients.

### SUMMARY OF THE INVENTION

The above-mentioned object is achieved by the invention providing a ventilator for supplying breathable gas to a patient, comprising:
an external housing;
an internal housing suspended within said external housing;
a gas flow generator located within said internal housing for creating a gas flow to the patient;
a gas inlet conduit extending between a first gas inlet opening in said external housing and a second gas inlet opening in said internal housing, and
a gas outlet conduit extending from a first gas outlet opening in the internal housing via a second gas outlet opening in the external housing and to a patient interface means adapted for introducing the breathable gas into the airway of said patient. The invention is especially characterized in that one or both of the gas inlet conduit and the gas outlet conduit exhibits:
a first substantially rigid conduit section, and
a second membrane conduit section having at least one membrane wall portion, said membrane wall portion separating a volume of breathable gas within the gas inlet conduit and/or gas outlet conduit from a volume of ambient air within the external housing.

In an advantageous embodiment of the invention, said membrane conduit section is formed with at least one flexible membrane wall portion and as a chamber, said chamber comprising a structural frame element which delimits said at least one flexible membrane wall portion, the latter being sealingly attached along its periphery to said structural frame element.

In one embodiment, the chamber is arranged on an exterior face of the internal housing, said exterior face defining an inner wall section of the chamber.

In an advantageous embodiment, a sound absorbent layer is provided within the chamber on the exterior face, in order to further improve the noise reducing characteristics of the ventilator.

In a well functioning embodiment, the structural frame comprises a grid with multiple grid apertures. The flexible membrane wall portion is here formed by a single membrane sheet which is attached to the grid at least along its periphery and covers said multiple grid apertures.

In another embodiment, the membrane conduit section is formed as a flexible tube section having a generally polyhedral cross-section, said flexible membrane wall portion being defined by the wall of said tube section. Advantageously, said flexible tube section is made of silicone rubber.

In a suitable embodiment, said first substantially rigid conduit section extends along the outline periphery of the external housing. Advantageously, the rigid conduit section is substantially L-shaped.

The external housing may be manufactured by molding, in such a way that the rigid conduit section is integrally formed with the external housing, and extends along the inside of an outer wall of said external housing.

The rigid conduit section is partially integrated in a hollow lift handle portion formed in the external housing, the first gas inlet opening being located in said lift handle portion.

In one embodiment, the internal housing is suspended in said external housing by means of one or more vibration isolator elements.

In a favorable embodiment, the structural frame element comprises a grid with multiple grid apertures, said flexible membrane wall portion being formed by a single membrane sheet which is attached to the grid at least along an outer periphery of the structural frame element and covers said multiple grid apertures. The grid apertures may for example be substantially rectangular.

Suitably, the chamber of the membrane conduit section is provided with a plurality of sound deflection barriers located between an entrance opening to the chamber and second inlet opening to the internal housing, said sound deflection barriers being arranged so as to at least partially block a direct sound propagation between said entrance opening and said second inlet opening.

In an advantageous embodiment, the gas flow generator is located in a sub housing within the internal housing, and a tortuous path, provided with a sound absorbing lining, is defined between the internal housing and said sub housing. The tortuous path extends between the second gas inlet opening in the internal housing and a third gas inlet opening in the sub housing. Preferably, the tortuous path is formed by successively arranged, and mutually displaced projecting barrier walls, wherein said sound absorbing lining is formed as at least one undulating plastic foam insert provided with slots for receiving said barrier walls.

In an alternative embodiment, the gas flow generator is likewise located in a sub housing within the internal housing, and a tortuous path, provided with successively arranged sound absorbing elements, is defined between the internal housing and said sub housing. Here, said sound absorbing elements are constituted by perforated metal plates coated with sound absorbing material on one or both sides thereof, said metal plates being of a uniform size and shape, and angled relative to a general direction of the tortuous path. Like in the above described embodiment, the tortuous path extends between the second gas inlet opening in the internal housing and a third gas inlet opening in the sub housing.

In an advantageous embodiment, said membrane wall portion is made of a thin plastic film.

The invention also provides a noise reduction method for a ventilator for supplying breathable gas to a patient, the ventilator comprising:
an external housing;
an internal housing suspended within said external housing;
a gas flow generator located within said internal housing for creating a gas flow to the patient;
a gas inlet conduit extending between a first gas inlet opening in said external housing and a second gas inlet opening in said internal housing, and
a gas outlet conduit extending from a first gas outlet opening in the internal housing via a second gas outlet opening in the external housing and to a patient interface means adapted for introducing the breathable gas into the airway of said patient. The method is especially characterized in that:
a volume (v) of breathable gas within the gas inlet conduit and/or gas outlet conduit is separated from a volume (V) of ambient air within the external housing, whilst allowing acoustic energy transfer between said volumes (v, V) by means of one or both of the gas inlet conduit and the gas outlet conduit exhibiting:
a first substantially rigid conduit section, and
a second membrane conduit section having at least one flexible membrane wall portion, said membrane wall portion allowing said acoustic energy transfer between said volumes (v, V).

The invention also provides a ventilator for supplying breathable gas to a patient, comprising:
an external housing;
an internal housing suspended within said external housing (4);
a gas flow generator located within said internal housing (6) for creating a gas flow to the patient;
a gas inlet conduit extending between a first gas inlet opening in said external housing and a second gas inlet opening in said internal housing, and
a gas outlet conduit extending from a first gas outlet opening in the internal housing via a second gas outlet opening in the external housing and to a patient interface means adapted for introducing the breathable gas into the airway of said patient, wherein one or both of the gas inlet conduit (22) and the gas outlet conduit exhibits:
a first substantially rigid conduit section, and
a second membrane conduit section, having at least one flexible membrane wall portion separating a volume (v) of breathable gas within the gas inlet conduit and/or gas outlet conduit from a volume (V) of ambient air within the external housing, whilst allowing acoustic energy transfer between said volumes (v, V).

The present invention enables a considerably more compact overall ventilator design, compared to what could physically be achieved with conventional noise reduction methods. Further features and advantages of the invention will be described in the detailed description of embodiments below.

### BRIEF DESCRIPTION OF THE DRAWINGS

The invention will now be described in greater detail by way of example only and with reference to the attached drawings, in which

FIG. 1 shows a schematic view of a ventilator according to a first exemplifying embodiment of the invention;

FIG. 2 shows a schematic view of a ventilator according to a second exemplifying embodiment of the invention;

FIG. 3 shows an exploded perspective view of the internal housing and the membrane conduit section formed thereupon;

FIG. 4 shows an alternative embodiment of the present invention, wherein the flexible membrane conduit section is formed as a flexible tube section;

FIG. 5 finally shows an enlarged cross-sectional view of a section of the tortuous path within the internal housing, and

FIG. 6 finally shows an alternative embodiment of the tortuous path within the internal housing. The tortuous path is here provided with successively arranged sound absorbing elements 86 constituted by perforated metal plates 88, coated with sound absorbing material.

### DESCRIPTION OF EXEMPLIFYING EMBODIMENTS

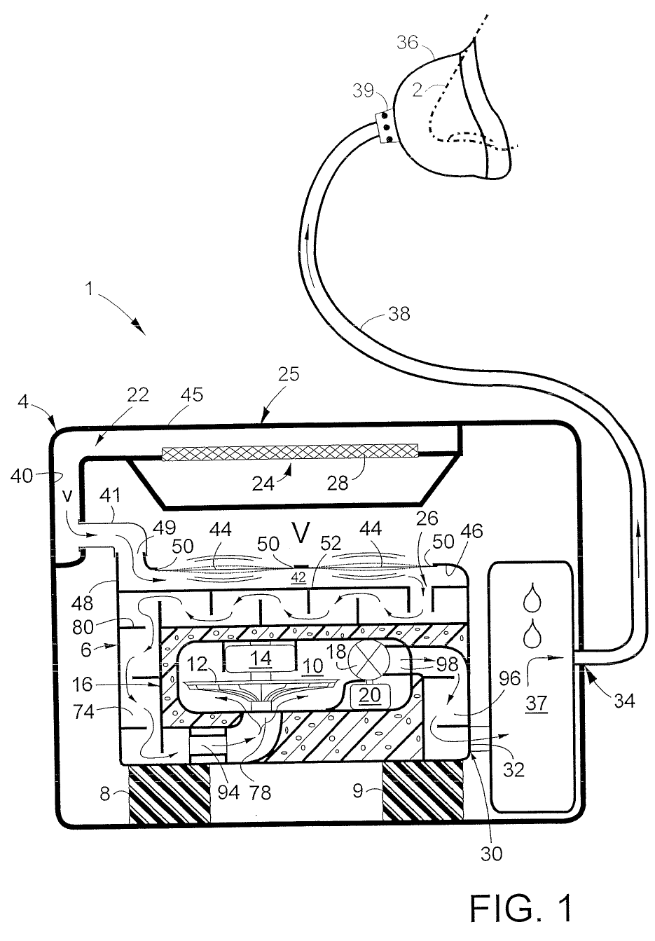

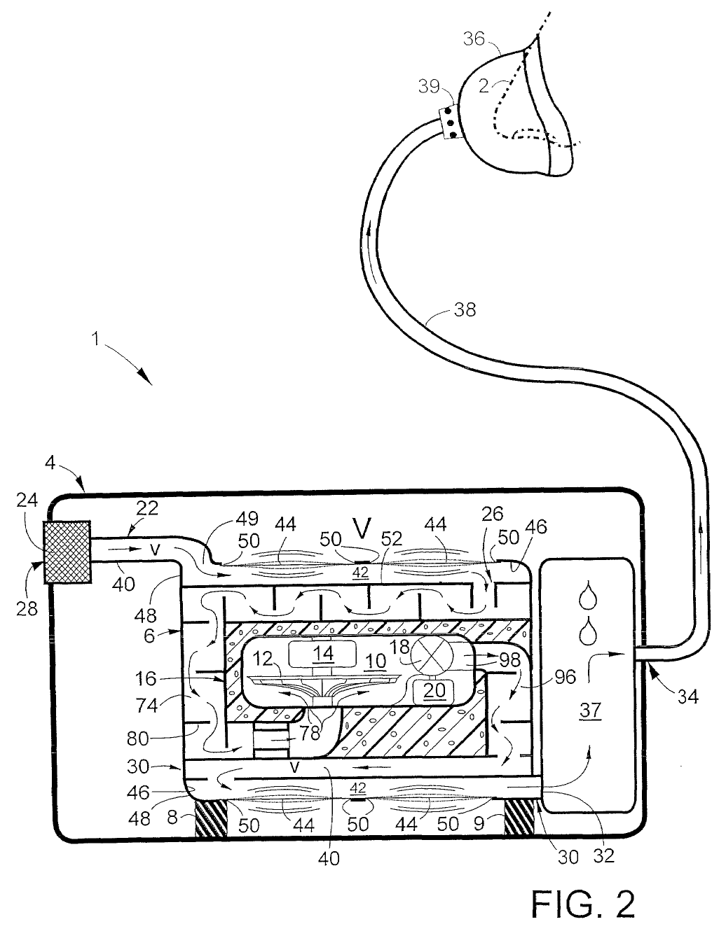

In FIG. 1, reference numeral 1 denotes a ventilator for supplying breathable gas—normally air—into the airway of a patient for treating breathing disorders such as for example Obstructive Sleep Apnea (OSA), Cheyne-Stokes respiration or emphysema. In the figure, a schematically drawn nose 2 of a patient is shown with dash-dotted lines. It should be noted that the schematic FIG. 1, as well as FIG. 2 described further later in this description, are both highly simplified and diagrammatic in order to clearly illustrate the basic features and principle operation of the invention. For example, all elements and conduits are shown oriented and extending in a common projection plane—i.e. the plane of the paper sheet—which is normally not the case in a production embodiment of the invention due to physical configuration requirements. Thus, a production embodiment of a ventilator 1 according to the invention may look significantly different than in FIGS. 1 and 2, although the basic features are still present as described.

As mentioned in the background section above, the invention is applicable to various types of medical ventilators 1. Thus, the ventilator 1 may for example be of the simplest CPAP-type (not shown) or it may be of either the Bi-Level CPAP type or the AutoCPAP-type.

The ventilator 1 comprises an external housing 4, which may advantageously be made of a suitably durable plastic material. To a person skilled in the art it will, however, be readily apparent that other suitable materials may be used alternatively. Examples of such materials include metals like steel, aluminum or zinc. Preferably, the external housing 4 is molded in two halves (not shown in FIGS. 1 and 2) for ease of assembly and service access.

An internal housing 6 is suspended within said external housing 4 by means of vibration isolator elements 8, for example consisting of blocks of a elastic material such as rubber or plastic materials with rubber-like properties. Furthermore, a gas flow generator 10 is located within the internal housing 6 for creating a gas flow to the patient. In a preferred embodiment, the gas flow generator 10 is constituted by an electric fan unit, comprising a fan rotor wheel 12 driven by an electric motor 14, as shown in a simplified, schematic view in FIG. 1. Alternatively, the gas flow generator 10 may theoretically be of a membrane-type or a bellows-type (as known per se, but not shown here), although the noise reduction features to be described below will be more effective with an electric fan unit. As described in the background above, a trend in modern ventilator technology is directed toward ever more compact and lightweight ventilators 1, that are unobtrusive at the bedside and offer increased mobility for patients. In order to achieve this, an electric fan unit is most often used as a gas flow generator 10 in modern ventilators 1.

In the embodiment shown in FIG. 1, the gas flow generator 10 is located in a sub housing 16 within the internal housing 6. This feature will be further described below, and is not an essential feature in the broadest sense of the present invention. The sub housing 16 further contains a control valve 18 adapted to control the airflow into the patients airway synchronized with the patients inspiratory cycle and expiratory cycle, respectively. The control valve 18 is driven by an electric stepper motor 20 and controlled by a control unit (not shown). Additionally, there is suitably a bypass conduit (not shown) extending from the control valve 18 back to the gas flow generator 10, for directing excess flow of breathable gas back into said gas flow generator 10 as the control valve 18 regulates the required gas flow to the patient. However, this bypass conduit is not shown here, since it would clutter the schematic illustration in FIG. 1. It should be noted that a CPAP ventilator in its simplest form is not provided with a control valve 18 at all, and that the invention is equally applicable to such a simple CPAP ventilator.

A gas inlet conduit 22 extends between a first gas inlet opening 24 in the external housing 4 and a second gas inlet opening 26 in said internal housing 6. A particle filter 28 is provided in the first inlet opening 24 in order to stop undesired particular matter from entering the ventilator 1.

Additionally, a gas outlet conduit 30 extends from a first gas outlet opening 32 in the internal housing 6 via a second gas outlet opening 34 in the external housing 6 and to a patient interface means 36 adapted for introducing the breathable gas into the airway of the patient. The shown embodiment also includes an air humidifier 37 located along the gas outlet conduit 30 between the first outlet opening 30 and the second outlet opening 34. The air humidifier 37 may be of a type well known per se and will thus not be further described in this description. Moreover, in the shown example, the patient interface means 36 includes a facial mask adapted for non-invasive attachment over the nose 3 of a patient.

As shown in FIGS. 1 and 2, the patient interface means 36 is also provided with exhaust apertures 39 for venting air exhaled by the patient into ambient air. Alternatively, the patient interface means 36 instead includes a tracheal tube (not shown) for invasive insertion in the trachea of a patient. The patient interface means 36 is connected to the second outlet opening 32 of the ventilator 1 by means of a flexible hose 38.

According to the main noise reduction feature of the present invention, one or both of the gas inlet conduit 22 and the gas outlet conduit 30 has a first substantially rigid conduit section 40, and a second membrane conduit section 42 having at least one flexible membrane wall portion 44. In the schematical cross-sectional view of FIG. 1, two membrane wall portions 44 are visible in the membrane conduit section 42. The membrane wall portions 44 separate a volume v of breathable gas within the gas inlet conduit 22 and/or gas outlet conduit 30 from a volume V of ambient air within the external housing 4, whilst allowing acoustic energy transfer between said volumes v, V. In an advantageous embodiment, the flexible membrane wall portions 44 are made as thin as possible whilst still being manageable in production and assembly process. The thickness of the membrane wall portions could be in the range size order of micrometer or millimetres, and may for example be a thin pliable plastic film. The plastic material may for example be polyester. A skilled person in the art will, however, recognize that the flexible membrane wall portions 44 may alternatively be made of other thin materials, such as silicone film or other films, foils or skins that will serve as a gas impermeable membrane.

In the embodiment shown in FIG. 1, the rigid conduit section 40 is substantially L-shaped and extends along the outline periphery of the external housing 4. The external housing 4 is manufactured by molding in such a way that the rigid conduit section 40 is integrally formed with the external housing 4, and extends along the inside of an outer wall 45 of said external housing 4. The L-shaped rigid conduit section 40, or rather one shank thereof, is partially integrated in a hollow lift handle portion 25 formed in the external housing 4, whereby the first gas inlet opening 24 is located in the lift handle portion 25. This design feature enables a more compact overall design of the ventilator 1.

Further, in the embodiment shown in FIG. 1, only the gas inlet conduit 22 is provided with a membrane conduit section 42 according to the invention. The membrane conduit section 42 offers substantial improvements in noise reduction characteristics in relation to the very compact size of the ventilator 1 as a whole, when compared to prior art ventilators with a conventional sound absorbing lining (not shown) within the external housing 6. In order to avoid noise transfer from the internal housing 6 to the external housing 4 via the gas inlet conduit 22, a vibration-isolating conduit section 41 is arranged between the rigid conduit section 22 and the membrane conduit section 42. Suitably, the vibration-isolating conduit section 41 is formed as a short tube made of flexible material such as silicone rubber or a material with similar characteristics.

If further noise reduction should be required, the gas outlet conduit 24 may also be provided with a membrane conduit section 42 having one or more membrane wall portions 44. Such an embodiment is schematically shown in FIG. 2, and it differs further from the previously shown embodiment in FIG. 1 in that the gas inlet conduit 22 is straight and not L-shaped as it is in FIG. 1.

In the embodiments shown in FIG. 1, the membrane conduit section 42 is formed as a chamber 46. The chamber 46 includes a structural frame element 48 adapted to support the flexible membrane wall portions 44. According to the schematical examples shown in FIGS. 1 and 2, a periphery 50 of each membrane wall portion 44 is attached to—or abuts—the structural frame element 48. The chamber 46 is arranged on an exterior face 52 of the internal housing 6, in such a way that the exterior face 52 defines an inner wall section 47 of the chamber 46. In an advantageous embodiment, a sound absorbent layer (not shown) is provided within the chamber 46 on the exterior face 52, in order to further improve the noise reducing characteristics of the ventilator 1. The sound absorbent layer may be of a plastic foam material or an equally suitable material, and may have multiple channels and projections (not shown).

According to the noise reduction method for the above described ventilator 1, a volume v of breathable gas within the gas inlet conduit 22 and/or gas outlet conduit 24 is separated from a volume V of ambient air within the external housing 4, whilst allowing acoustic energy transfer between said volumes v, V, by means of said first substantially rigid conduit section 40, and the second membrane conduit section 42 having at least one flexible membrane wall portion 44. Thus, the membrane wall portion (or portions) 44 allows acoustic energy transfer between said volumes v, V.

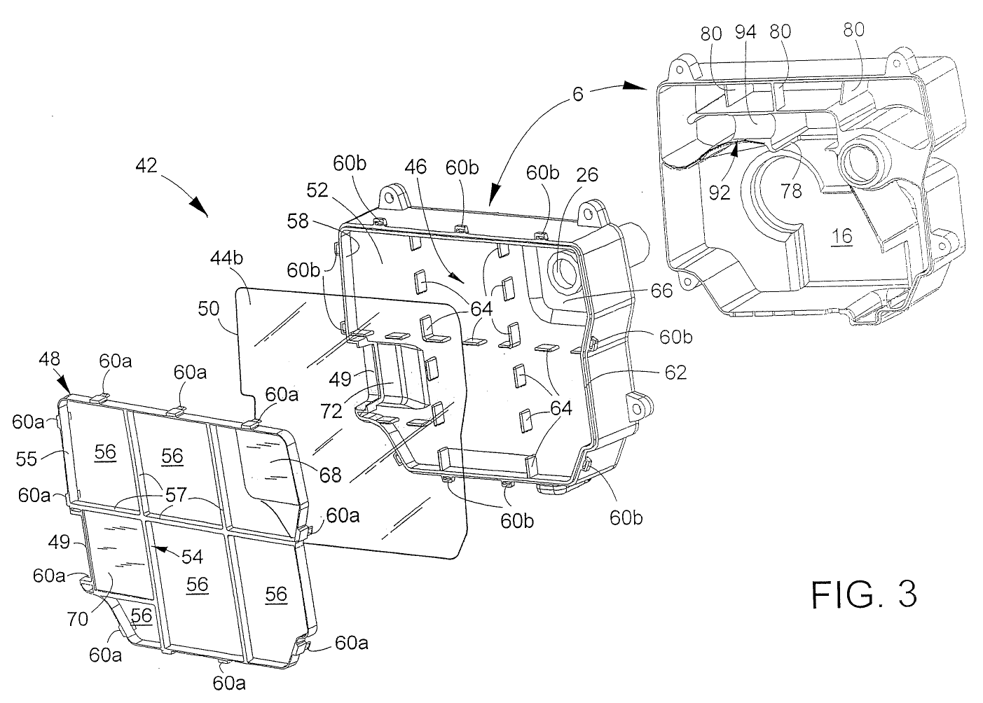

In the embodiment shown in the exploded view of FIG. 3, the structural frame element 48 comprises a grid 54 with multiple grid apertures 56 formed by a plurality of reinforcement crossbars 57. Here, multiple flexible membrane wall portions 44 are defined within the grid 54 by its grid apertures 56 by using a single membrane sheet 44b, which is attached to the grid 54 along an outer periphery 55 of the structural frame element 48 so that it fully covers the multiple grid apertures 56. In the shown embodiment, the internal housing 6 is provided with a projecting circumferential side wall 58, which partly defines the chamber 46 of the membrane conduit section 42. In the shown embodiment, the structural frame element 48 is attached to the internal housing 6 by means of a plurality of snap fasteners 60a, 60b located along its outer periphery 55 and along the sidewall 58 on the internal housing 6, respectively. Alternatively, the structural frame element 48 may be attached to the internal housing 6 by means of screws, clamps or other suitable fastening means (not shown). As seen in the exploded view, the single membrane sheet 44b is arranged to be clamped between the periphery 55 of the structural frame element 48 and a guide rim 62 for the structural frame element 48 on the sidewall 58. As seen in FIG. 3, the grid apertures 56 are substantially rectangularly shaped. However, the skilled man in the art will easily recognize that the shape of these grid openings 56 may be varied in a great many ways. Hence, for example, the grid openings 56 may alternatively be circular, trapezoidal, triangular, oval, rhombus, or any combination thereof.

With further reference to FIG. 3, the chamber 46 is provided with a plurality of sound deflection barriers 64 located between an entrance opening 49 to the chamber 46 and second inlet opening 26 to the internal housing 6. The sound deflection barriers 64 are arranged so as to at least partially block direct sound propagation between said entrance opening 49 and said second inlet opening 26. In this way, noise reduction is further improved since sound waves within the chamber 46 are deflected in several main propagation directions and do not “see” a direct route to the inlet opening 26. As shown in FIG. 3, the sound deflection barriers 64 extend both horizontally and vertically within the chamber 46, and may alternatively also extend diagonally or at various other angles (not shown). A recess 66 is defined around the second gas inlet opening 26, and a corresponding flat rigid flow stabilization plate 68 is integrally formed in the structural frame element 48 directly above the recess 66, in order to locally stabilize the gas flow in the immediate vicinity of the second gas inlet opening 26. Correspondingly, a similar flat rigid flow stabilization plate 70 is integrally formed in the structural frame element 48 directly above a recess 72 for the entrance opening 49 to the chamber 46.

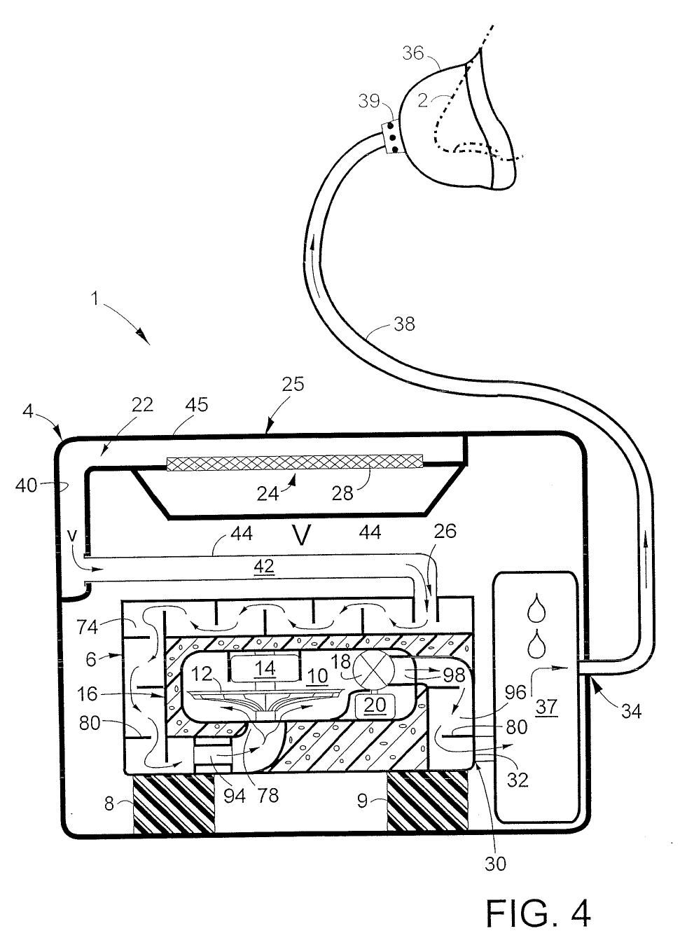

FIG. 4 illustrates an alternative embodiment of the present invention, wherein the flexible membrane conduit section 44 is formed as a flexible tube section. The flexible membrane wall portion 44 is here defined by the wall of said tube section. The flexible tube section is suitably of silicone rubber or a flexible material with similar characteristics, and preferably exhibits a rectangular or otherwise polyhedral cross-section rather than a circular cross-section, so as to maximize the degree of mobility of the membrane wall portion 44.

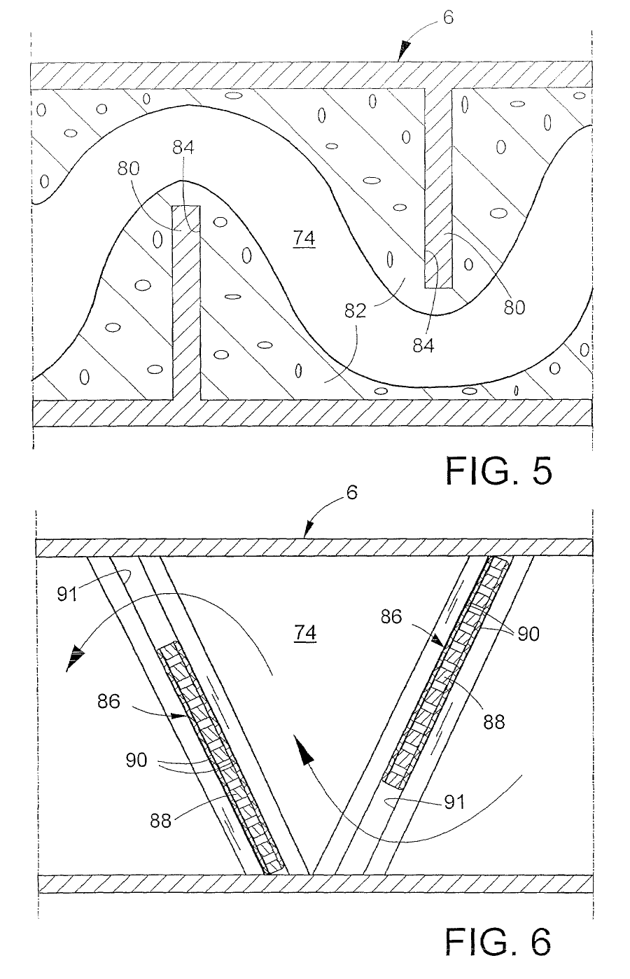

In the embodiment shown in FIG. 1 and FIGS. 3-5, a tortuous path 74 is defined between the internal housing 6 and said sub housing 16. The tortuous path 74 is provided with a sound absorbing lining 76 and extends between the second gas inlet opening 26 in the internal housing 6 and a third gas inlet opening 78 in the sub housing 16. More particularly, the tortuous path 74 exhibits successively arranged, and mutually displaced projecting barrier walls 80. As shown in the enlarged partial cross-sectional view of FIG. 5, the sound absorbing lining 76 is formed as at least one undulating plastic foam insert 82 provided with slots 84 for receiving said barrier walls 80. In the exploded view of FIG. 3, the tortuous path 74 is shown without plastic foam inserts 82 in order to illustrate the route of the tortuous path 74 and the extension of the barrier walls 80. Nor does FIG. 4 show the sub housing 16 for the gas flow generator 10. At an end portion 92 of the tortuous path 74, immediately before the gas inlet opening 78, is a straight cylindrical passage 94 intended to provide an acoustic mass volume for further dampening of structural and dynamic noise from the gas flow generator 10 (not shown in FIG. 3). As an optional additional noise reducing feature, a shorter second tortuous path 96 is located between an inner gas outlet opening 98 from the sub housing 16 and the gas outlet opening 32 in the internal housing 6.

FIG. 6 shows an alternative embodiment of the tortuous path 74 within the internal housing 6. The tortuous path 74 is here provided with successively arranged sound absorbing elements 86 constituted by perforated metal plates 88, coated with sound absorbing material 90 on one or both sides thereof. The metal plates 88 are preferably generally rectangular and are all of a uniform size and shape. The sound absorbing elements 86 are inserted in angled mounting slots 91 and are thus angled relative to a general direction of the tortuous path 74. Like in the above described embodiment, the tortuous path 74 extends between the second gas inlet opening 26 in the internal housing 6 and a third gas inlet opening 78 in the sub housing 16 (not shown in this embodiment).

It is to be understood that the invention is by no means limited to the embodiments described above, and may be varied freely within the scope of the appended claims. For example, in an alternative, not shown embodiment, the structural frame element 48 may itself be made of a flexible material and is integrally formed with at least one flexible membrane wall portion 44. However, in such an embodiment, the thickness and structural stiffness of the structural frame element 48 substantially exceeds the thickness of the flexible membrane wall portion 44. In a version of this embodiment, provided with multiple membrane wall portions 44, the previously mentioned grid 54 serves as shape retaining reinforcement adapted to maintain the shape of the chamber 46. Although this embodiment is not explicitly shown, it would essentially correspond to the appearance of the embodiment in FIG. 1. Optionally, the vibration-isolating conduit section 41 may also be integrally formed with the structural frame element 48. Hence, in such an embodiment, the structural frame element 48, the membrane wall portions 44 and the vibration-isolating conduit section 41 may be conveniently molded as a single unit in, for example, a silicone rubber material.

### LIST OF REFERENCE NUMERALS AND SIGNS

1. Ventilator
2. Schematic illustration of a patients nose
4. External housing
6. Internal housing
8. Vibration isolator elements
10. Gas flow generator
12. Fan rotor wheel
14. Electric motor
16. Sub housing for gas flow generator
18. Control Valve
20. Stepper motor
22. Gas inlet conduit
24. First gas inlet opening
25. Lift handle portion
26. Second gas inlet opening
28. Particle filter
30. Gas outlet conduit
32. First gas outlet opening
34. Second gas outlet opening
36. Patient interface means
37. Air humidifier
38. Flexible hose
39. Exhaust apertures
40. Rigid conduit section
41. Vibration-isolating conduit section
42. Membrane conduit section
44. Flexible membrane wall portion
44b. Single membrane sheet
45. Outer wall of external housing
46. Chamber formed in membrane conduit section
47. Inner wall section of chamber
48. Structural frame element
49. Entrance opening to chamber in membrane conduit section
50. Periphery of the flexible membrane wall portion
52. Exterior face of the internal housing
54. Grid
55. Outer periphery of grid
56. Grid apertures
57. Reinforcement crossbars
58. Side wall of membrane conduit section
60a. Snap fasteners, male
60b. Snap fasteners, female
62. Guide rim
64. Sound deflection barriers
66. Recess around second gas inlet opening
68. Flat rigid flow stabilization plate
70. Flat rigid flow stabilization plate
72. Recess for entrance opening to the chamber
74. Tortuous path in internal housing
76. Sound absorbing lining
78. Third gas inlet opening in the sub housing
80. Barrier walls in tortuous path
82. Undulating plastic foam insert
84. Slots in foam insert for receiving barrier walls
86. Sound absorbing elements (in alternative embodiment of FIG. 6)
88. Perforated metal plates (in alternative embodiment of FIG. 6)
90. Sound absorbing material (in alternative embodiment of FIG. 6)
91. Angled mounting slots (in alternative embodiment of FIG. 6)
92. End portion of the tortuous path
94. Straight cylindrical passage
96. Second tortuous path
98. Outlet opening from sub housing

1. A ventilator for supplying breathable gas to a patient, comprising:
an external housing;
an internal housing suspended within said external housing;
a gas flow generator located within said internal housing for creating a gas flow to the patient;
a gas inlet conduit extending between a first gas inlet opening in said external housing and a second gas inlet opening in said internal housing, and
a gas outlet conduit extending from a first gas outlet opening in the internal housing via a second gas outlet opening in the external housing and to a patient interface means adapted for introducing the breathable gas into the airway of said patient, wherein one or both of the gas inlet conduit and the gas outlet conduit exhibits:
a first substantially rigid conduit section, and
a second membrane conduit section, with a membrane wall portion separating a volume of breathable gas within the gas inlet conduit and/or gas outlet conduit from a volume of ambient air within the external housing.

2. The ventilator according to claim 1, wherein said membrane conduit section is formed with at least one flexible wall portion and as a chamber, said chamber comprising a structural frame element which delimits said at least one flexible membrane wall portion.

3. The ventilator according to claim 2, wherein said chamber is arranged on an exterior face of the internal housing, said exterior face defining an inner wall section of the chamber.

4. The ventilator according to claim 3, wherein a sound absorbent layer is provided within said chamber on said exterior face.

5. The ventilator according to claim 2, wherein said structural frame element comprises a grid with multiple grid apertures, said flexible membrane wall portion being formed by a single membrane sheet which is attached to the grid at least along an outer periphery of the structural frame element and covers said multiple grid apertures.

6. The ventilator according to claim 5, wherein said grid apertures are substantially rectangular.

7. The ventilator according to claim 1, wherein said chamber is provided with a plurality of sound deflection barriers located between an entrance opening to the chamber and second inlet opening to the internal housing, said sound deflection barriers being arranged so as to at least partially block direct sound propagation between said entrance opening and said second inlet opening.

8. The ventilator according to claim 1, wherein a flexible vibration-isolating conduit section is arranged between the rigid conduit section and the membrane conduit section.

9. The ventilator according to claim 1, wherein said membrane conduit section is formed as a flexible tube section having a generally polyhedral cross-section, said flexible membrane wall portion being defined by the wall of said tube section.

10. The ventilator according to claim 9, wherein said flexible tube section is made of silicone rubber.

11. The ventilator according to claim 1, wherein said first substantially rigid conduit section extends along the outline periphery of the external housing.

12. The ventilator according to claim 11, wherein the rigid conduit section is substantially L-shaped.

13. The ventilator according to claim 12, wherein the external housing is manufactured by molding, wherein the rigid conduit section is integrally formed with the external housing, and extends along the inside of an outer wall of said external housing.

14. The ventilator according to claim 13, wherein the rigid conduit section is partially integrated in a hollow lift handle portion formed in the external housing, the first gas inlet opening being located in said lift handle portion.

15. The ventilator according to claim 11, wherein the external housing is manufactured by molding, wherein the rigid conduit section is integrally formed with the external housing, and extends along the inside of an outer wall of said external housing.

16. The ventilator according to claim 15, wherein the rigid conduit section is partially integrated in a hollow lift handle portion formed in the external housing, the first gas inlet opening being located in said lift handle portion.

17. The ventilator according to claim 1, wherein said internal housing is suspended in said external housing by means of one or more vibration isolator elements.

18. The ventilator according to claim 1, wherein:
said gas flow generator is located in a sub housing within the internal housing, and
a tortuous path, provided with a sound absorbing lining, is defined between the internal housing and said sub housing, said tortuous path extending between the second gas inlet opening in the internal housing and a third gas inlet opening in the sub housing.

19. The ventilator according to claim 18, wherein said tortuous path is formed by successively arranged, and mutually displaced projecting barrier walls, wherein said sound absorbing lining is formed as at least one undulating plastic foam insert provided with slots for receiving said barrier walls.

20. The ventilator according to claim 1, wherein:
said gas flow generator is located in a sub housing within the internal housing, and
a tortuous path, provided with successively arranged sound absorbing elements, is defined between the internal housing and said sub housing,
said sound absorbing elements being constituted by perforated metal plates coated with sound absorbing material on one or both sides thereof, said metal plates being of a uniform size and shape, and angled relative to a general direction of the tortuous path, and
said tortuous path extending between the second gas inlet opening in the internal housing and a third gas inlet opening in the sub housing.

21. The ventilator according to claim 1, wherein said membrane wall portion is made of a thin plastic film.

22. A noise reduction method for a ventilator for supplying breathable gas to a patient, the ventilator comprising:
an external housing;
an internal housing suspended within said external housing;
a gas flow generator located within said internal housing for creating a gas flow to the patient;
a gas inlet conduit extending between a first gas inlet opening in said external housing and a second gas inlet opening in said internal housing, and
a gas outlet conduit extending from a first gas outlet opening in the internal housing via a second gas outlet opening in the external housing and to a patient interface means adapted for introducing the breathable gas into the airway of said patient, wherein
a volume of breathable gas within the gas inlet conduit and/or gas outlet conduit is separated from a volume of ambient air within the external housing, whilst allowing acoustic energy transfer between said volumes by means of one or both of the gas inlet conduit and the gas outlet conduit exhibiting:
a first substantially rigid conduit section, and
a second membrane conduit section having at least one flexible membrane wall portion, said membrane wall portion allowing said acoustic energy transfer between said volumes.

23. A ventilator for supplying breathable gas to a patient, comprising:
an external housing;
an internal housing suspended within said external housing;
a gas flow generator located within said internal housing for creating a gas flow to the patient;
a gas inlet conduit extending between a first gas inlet opening in said external housing and a second gas inlet opening in said internal housing, and
a gas outlet conduit extending from a first gas outlet opening in the internal housing via a second gas outlet opening in the external housing and to a patient interface means adapted for introducing the breathable gas into the airway of said patient, wherein one or both of the gas inlet conduit and the gas outlet conduit exhibits:
a first substantially rigid conduit section, and
a second membrane conduit section, having at least one flexible membrane wall portion separating a volume of breathable gas within the gas inlet conduit and/or gas outlet conduit from a volume of ambient air within the external housing, whilst allowing acoustic energy transfer between said volumes.
